(19) United States
(12) Patent Application Publication
(10) Pub. No.: US 2024/0301392 A1
(43) Pub. Date: Sep. 12, 2024

(54) HOMOGENEOUS METHOD TO PREPARE SPERM DNA FROM SEXUAL ASSAULT CASES

(71) Applicant: Alex Garvin, Brustatt (FR)

(72) Inventor: Alex Garvin, Brustatt (FR)

(21) Appl. No.: 18/655,262

(22) Filed: May 5, 2024

Related U.S. Application Data
(63) Continuation of application No. 18/451,974, filed on Aug. 18, 2023.
Application No. 18/451,974 is a continuation of application No. 12/008,385, filed on Jan. 11, 2008, now Pat. No. 11,781,127.
(60) Provisional application No. 60/880,497, filed on Jan. 16, 2007.

(51) Int. Cl. C12N 15/10

(52) U.S. Cl. CPC C12N 15/1003

(57) ABSTRACT

The present invention provides a method for the isolation of sperm DNA from swabs taken from rape victims without having to perform a change in buffers. Non-sperm cells from the victim are digested with an enzyme and solubilized, and then in the same buffer an enzyme capable of digesting soluble DNA is added and the victim's DNA is degraded, leaving only the rapist's DNA intact. Since no change of buffer is needed, no centrifugation or filtration steps are needed. The inventive method has utility particularly in the forensic science field.

### FIELD OF THE INVENTION

The present invention relates to isolation of sperm DNA from a mixture of sperm and non-sperm cells. The inventive method has utility in forensic analyses.

### BACKGROUND

Police departments in the United States currently have a backlog of as many as 500,000 unprocessed swabs taken from rape victims, and the identity of the rapist can be determined by profiling the sperm DNA present on these swabs. Sperm are normally obtained from a rape victim by rubbing a swab against a mucous membrane, resulting in large numbers of the victim's epithelial cells being collected with the rapist's sperm, and these epithelial cells contain large amounts of the victim's DNA. The victim's DNA acts as a contaminant that must be removed from the sperm prior to purification and analysis of the sperm DNA.

The standard method for purifying sperm from vaginal swabs is to first resuspend all cells from the swab and to selectively digest the victim's epithelial cells with a solution containing Proteinase K and SDS (sodium dodecyl sulfate). Sperm nuclei are impervious to this treatment because they have disulfide bond cross-linked thiol-rich proteins, while other cell types are digested and the corresponding DNA is solubilized. The intact sperm are separated from the solubilized, contaminating DNA by centrifugation, careful removal of supernatant, and extensive washing of the sperm pellet (see e.g., Giusti et al., J Forensic Sci., 31:409-417, 1986; Gill et al. Nature 318:577-579, 1985; Wiegand et al., Int J Legal Med, 104:359-360, 1992; and Yoshida et al., Forensic Sci Int., 72:25-33, 1995). Unfortunately, the processes of centrifugation and careful removal of supernatant are difficult to automate, labor intensive, and result in the loss of some sperm.

Uchiyama et al (Japanese Journal of Forensic Science and Technology Volume 11, Number 1, p 105-112, 2006) show that the sperm pellet washing steps can be avoided by selectively degrading the victim's DNA with DNAse I following digestion of the epithelial cells with proteinase K/SDS, pelleting of the sperm, and removal of the supernatant, and resuspension of the sperm pellet in a DNAse I buffer (see FIG. 1 of paper). While this group found that DNAse I digests soluble DNA and does not digest the sperm DNA (an essential component of the present invention), they did not attempt to develop a centrifugation-free method, namely they did not consider using a buffer that would allow Proteinase K to digest the epithelial cells and DNAse I to digest the solubilized DNA. Since DNAse I is not active in SDS, this approach requires a buffer change, which requires centrifugation. All prior art that uses a detergent, including the Uchiyama paper, uses an SDS or Sarkosyl containing buffer for the proteinase K lysis step, and this type of buffer is incompatible with DNAse I digestion of the solubilized DNA, unless the SDS is removed.

Chen et al. (J Forensic Science 43:114-118, 1998) propose to separate the sperm from the epithelial cells before preferential lysis by gravitational or mild vacuum filtration through a 5-10 micron nylon mesh membrane which is supposed to retain the epithelial cells while the sperm pass through. DNA is then prepared from the sperm collected in the filtrate. Unfortunately the pores of these filters will expand under pressure requiring that only gravity be used as the driving force to minimize the unwanted passage of epithelial cells. In the absence of a strong driving force, capillary action on the filter surface competes with gravity flow through the filter and results in a large retention volume and difficulties with sample handling (present applicant's observation). Furthermore, DNA from epithelial cells lysed by the harsh detergent required for efficient cell re-suspension will pass through the filter with intact sperm.

Garvin (J Forensic Science 48:1084-7, 2003) and Bille (US patent application 20030215845) showed that sperm could be separated from digested epithelial cell DNA by collecting the sperm on a filter while allowing the solubilized DNA to pass through the filter. Although this method avoids centrifugation, it still requires a separation step, and the sperm DNA must be eluted from the filter, a process that can result in sperm DNA loss.

Attempts have also been made to use anti-sperm antibody coated magnetic beads (Eisenberg, A. J. “Development of a Spermatozoa Capture System for the Differential Extraction of Sexual Assault Evidence”; paper presented at: Profiling PCR and Beyond Conference, 2002; Washington, D.C.). Epitope stability, however, is a likely problem with this approach when applied to casework, because detergents are required to efficiently elute sperm from the swabs and these detergents destroy most of the epitopes recognized by the anti-sperm antibodies. Magnetic beads have been successfully used for many cell separation applications (Haukanes & Kvam, Biotechnology (N Y). 11:60-63, 1993), but it remains to be seen if they can be used to separate human cells that have been dried onto an adsorbent substrate and then resuspended.

A number of attempts have been made to circumvent the selective lysis process. For example, Y chromosome polymorphic markers can be amplified from unfractionated swab DNA (Sibille, et al., Forensic Sci Int. 125:212-216, 2002). However the data provided cannot be used to probe the autosomal STR profiles in the FBI CODIS database, it won't work when the rape victim is male, and males of the same paternal lineage usually have identical Y chromosome STR patterns.

Another approach towards avoiding selective lysis is to physically separate sperm from intact epithelial cells. This has been done by flow cytometry (Schoell et al., Obstet Gynecol. 94:623-627, 1999), however this technique is inherently slow due to the need to analyze and sort one cell at a time and is unlikely to be applied to casework.

### SUMMARY OF THE INVENTION

The present invention provides a method for the isolation of sperm DNA from samples having at least one other cell type. The method is based on selective degradation of non-sperm cells and then, in essentially the same buffer, degradation of the DNA that was contained within these cells by treatment with a DNA degrading agent that selectively degrades non-sperm cell DNA. The inventive method has utility in the forensics field.

Preferred aspects provide an identification method, comprising: obtaining sperm DNA according to the above described methods; and determining an identity for the sperm donor, based on the isolated sperm DNA. Preferably, the identity determined is that of a perpetrator of a sexual assault.

The inventive method is applicable in the forensic setting for high throughput processing of samples, such as swabs from sexual assault victims, to obtain a sperm DNA profile, wherein such swabs contain in addition to a relatively small amount of sperm a much larger amount of the victim's epithelial cells and possibly blood cells.

Therefore, it is an object of the invention to create a method for processing samples containing a mixture of sperm and other cells (in particular epithelial cells or blood cells) with the aim of analyzing the sperm DNA contained in the sample. The preferred method is fast, suitable for automation, and provides reliable results even when the amount of sperm present in a test sample is much smaller than the amount of non-sperm cells.

### BRIEF DESCRIPTION OF THE DRAWINGS

FIG. 1 shows an operator buccal control fraction made from buccal DNA.

FIG. 2 shows a male buccal control fraction made from male buccal DNA.

FIG. 3 shows a female buccal control fraction made from female buccal DNA.

FIG. 4 shows post-coital vaginal swabs taken at twenty-four hours after coitus.

FIG. 5 shows post-coital vaginal swabs taken at thirty-two hours after coitus.

FIG. 6 shows post-coital vaginal swabs taken at forty hours after coitus.

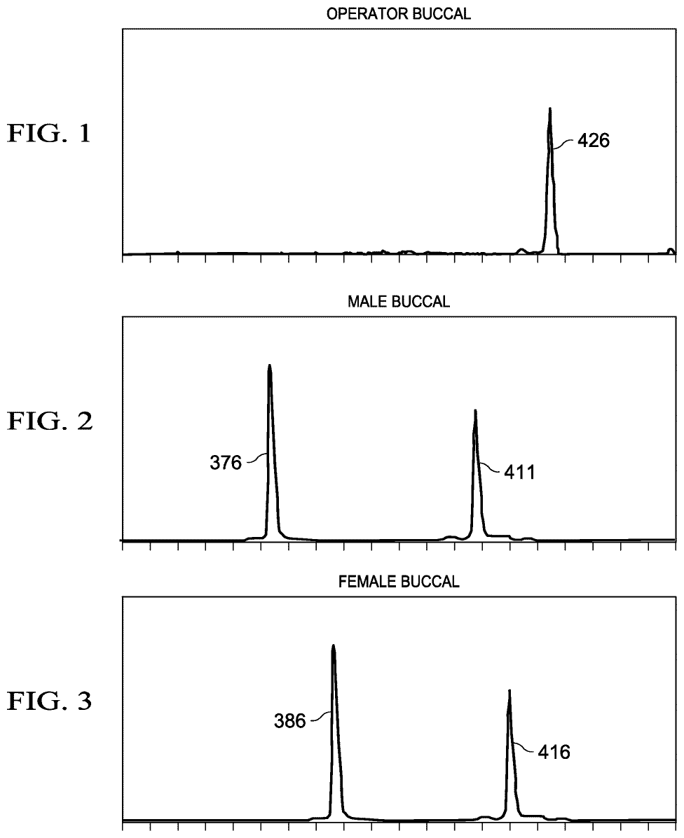

### DETAILED DESCRIPTION OF THE INVENTION

A sample of sperm and non-sperm cells present on a swab must be resuspended before DNA can be isolated. The standard method is to use a 2% SDS/proteinase K solution to do this. The present invention proposes instead to use a high concentration of an effective detergent, plus proteinase K, to do the same thing, however, the detergent must be compatible with downstream processes, namely digestion of soluble DNA with DNAse I. Therefore a different detergent must be used. Triton X-100 at 2% concentration is an effective detergent that can solubilize proteins and DNA, yet DNAse I is active in Triton X-100 at 2%, while it is not active in 2% SDS. The first step is to resuspend the cells from the swab cutting in a 500 μl solution containing: 2% Triton X-100, 20 mM Tris pH 8.0, and proteinase K at 400 ug/ml. Tris pH 8.0 is to insure that the buffer is not at an acidic pH (which will degrade DNA) and Proteinase K is required to digest the non-sperm cells. The swab cutting in vortexed 10 seconds and then incubated in this buffer for 2 hours at 56 degrees. A 10% aliquot (50 ul) of the solution is removed to obtain the victim's DNA fraction.

The next step is to degrade the solubilized non-sperm DNA without having to change the buffer. This is done by transferring 350 ul of the ProK digested material to a fresh tube (the swab cutting is left behind) and adding: 6 mM CaCl2, 6 mM MgCl2, and 360 units of DNAse I. The calcium and magnesium are added to activate the DNAse I enzyme. The DNAse I is allowed to digest soluble DNA by incubation at 56 degrees for 1 hour. Importantly, the DNAse I is active in the buffer that it is placed in, which would not be the case if SDS were in the buffer.

The next step is to inactivate the nuclease, and to lyse the sperm. This is done by adding:

25 mM EDTA (ethylene diamine tetra acidic acid), and 50 mM DTT (dithiothreitol). Incubation is for 5 minutes at 56 degrees.

The sperm DNA is now in solution and free of non-sperm DNA, and ready for further purification. The DNA is in a buffer that is compatible with many down-stream purification methods, such as guanidinium/magnetic silica beads and charge/switch magnetic beads.

### EXAMPLES

50,000 sperm were treated using the above protocol, either with or without DNAse 1 treatment. DNA was isolated using Qiagen microamp columns, and resuspended in 20 μl of AE buffer. The DNA was quantified using a qPCR assay capable of detecting sub-picogram amounts of DNA. The qPCR assay uses a SyberGreen dye and primers to amplify the 18S ribosome genes. The results are as follows:
No DNase: 42 nanograms
Plus DNase: 39 nanograms

These results show that DNAse 1 treatment does no degrade sperm DNA.

Epithelial cells from a buccal swab were then digested with 1 ml 2% Triton X100, 20 mM Tris pH 8.0, and proteinase K at 400 μg/ml for 4 hours at 56 degrees, and this solution was split in half. Half was treated with DNAse 1 as described above and half was not treated with DNAse 1. The DNA was isolated using Qiagen microamp columns, resuspended in 20 μl of AE buffer, and quantitated by qPCR. The results are as follows:
No DNase: 340 nanograms
Plus DNase 300 picograms

Therefore DNase 1 treatment can reduce the amount of epithelial cell DNA by over 1000 fold.

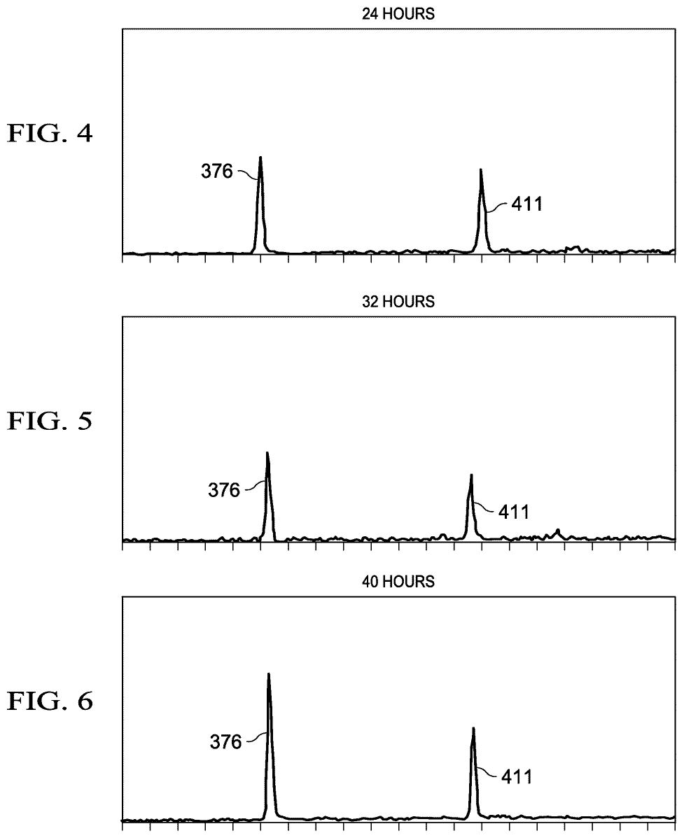

Post-coital vaginal swabs were treated with the nuclease protocol and profiled using the PowerPlex 16 kit from Promega. As shown in FIGS. 4-6, the male fraction DNA obtained from swabs taken at 24, 32, and 40 hours give a clear male profile for the Penta E locus.

1. A method for purifying DNA from intact sperm in sexual assault samples without a centrifugation step comprising:
a) obtaining a sexual assault sample and placing the sample contents in a buffer compatible with enzymes that can digest non-sperm cells and soluble DNA, said enzymes being a first enzyme that can digest non-sperm cells and said a second enzyme that can digest soluable DNA,
b) incubating with said first enzyme to obtain soluble non-sperm DNA and intact sperm,
c) incubating with said second enzyme to digest soluble non-sperm DNA, and
d) purifying for further analysis DNA from any intact sperm present in the sample,
wherein the selective digesting of non-sperm cells and non-sperm DNA without digesting sperm DNA is accomplished without a centrifugation step.

2. The method according to claim 1, wherein the buffer does not contain Sodium Dodecyl Sulfate.

3. The method according to claim 1, wherein the buffer contains Triton X-100.

4. The method according to claim 1, wherein the first enzyme is Proteinase K.

5. The method according to claim 1, wherein the second enzyme is DNase-I.

6. The method according to claim 1, wherein the sample is obtained from a victim of sexual assault.

7. An identification method, comprising: obtaining sperm DNA according to the method of claim 1; and determining an identity for the sperm donor, based, at least in part, on the isolated sperm DNA.

8. The method of claim 7, wherein the identity determined is that of a perpetrator of a sexual assault.
